(19) United States
(12) Patent Application Publication
(10) Pub. No.: US 2014/0171819 A1
(43) Pub. Date: Jun. 19, 2014

(54) Neurological Monitoring Method and System

(71) Applicant: Timothy Patterson, Pleasanton, CA (US)

(72) Inventor: Timothy Patterson, Pleasanton, CA (US)

(21) Appl. No.: 13/714,367

(22) Filed: Dec. 13, 2012

(51) Int. Cl. A61B 5/0476

(52) U.S. Cl. CPC A61B 5/0476
USPC 600/544; 600/300

(57) ABSTRACT

A neurological monitoring method and system that allows a user to monitor a neurological organ while recovering from a neurological injury. The neurological monitoring method allows the user to recover from the injury, and an invited follower to map the neurological organ and study the neurological organ from a display device. The method includes sensing the organ with a sensor, such as EEG electrodes. A signal generated by the neurological organ is then recorded by the sensor and transmitted to data storage, including a cloud. The signal can be reconfigured and retransmitted to a display device. The signal includes Hertz and real time frequencies. The signal includes a signal parameter with a high range and a low range. An alert actuates when the signal exceeds the parameter. The signal is monitored by the user and invited followers for better understanding the condition of the neurological organ.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to neurology. More particularly, one or more embodiments of the invention relate to a method of monitoring neurology.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that neurology is a medical specialty dealing with disorders of the nervous system, including the diagnosis and treatment of all categories of disease involving the central, peripheral, and autonomic nervous systems, and their coverings, blood vessels, and all effector tissue, such as muscle

Typically, traumatic brain injury (TBI) occurs when an external force traumatically injures the brain. TBI can be classified based on severity, mechanism (closed or penetrating head injury), or other features. TBI can cause a host of physical, cognitive, social, emotional, and behavioral effects, and outcome can range from complete recovery to permanent disability or death.

Typically, electroencephalography (EEG) is the recording of electrical activity along the scalp. EEG measures voltage fluctuations resulting from ionic current flows within the neurons of the brain.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates a flowchart diagram of the steps for an exemplary neurological monitoring method, in accordance with an embodiment of the present invention;

FIG. 2 illustrates a block diagram for an exemplary neurological monitoring system, in accordance with an embodiment of the present invention;

FIG. 3 illustrates a top view of an exemplary display portion for a user, in accordance with an embodiment of the present invention;

FIGS. 4A, 4B, and 4C illustrate a top view of an exemplary display portion for a follower, wherein FIG. 4A illustrates an exemplary alert portion set for the follower and the user, FIG. 4B illustrates an exemplary alert portion set by the follower to track the user, and FIG. 4C illustrates an exemplary desktop display portion used by the follower, in accordance with an embodiment of the present invention; and

FIG. 5 illustrates a typical computer system that, when appropriately configured or designed, can serve as a computer system in which the invention may be embodied, in accordance with an embodiment of the present invention.

Unless otherwise indicated illustrations in the figures are not necessarily drawn to scale.

### DETAILED DESCRIPTION OF SOME EMBODIMENTS

Embodiments of the present invention are best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

“Software” may refer to prescribed rules to operate a computer. Examples of software may include: code segments in one or more computer-readable languages; graphical and or/textual instructions; applets; pre-compiled code; interpreted code; compiled code; and computer programs.

A “computer-readable medium” may refer to any storage device used for storing data accessible by a computer. Examples of a computer-readable medium may include: a magnetic hard disk; a floppy disk; an optical disk, such as a CD-ROM and a DVD; a magnetic tape; a flash memory; a memory chip; and/or other types of media that can store machine-readable instructions thereon.

A “computer system” may refer to a system having one or more computers, where each computer may include a computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet.

Exemplary networks may operate with any of a number of protocols, such as Internet protocol (IP), asynchronous transfer mode (ATM), and/or synchronous optical network (SONET), user datagram protocol (UDP), IEEE 802.x, etc.

Embodiments of the present invention may include apparatuses for performing the operations disclosed herein. An apparatus may be specially constructed for the desired purposes, or it may comprise a general-purpose device selectively activated or reconfigured by a program stored in the device.

Embodiments of the invention may also be implemented in one or a combination of hardware, firmware, and software. They may be implemented as instructions stored on a machine-readable medium, which may be read and executed by a computing platform to perform the operations described herein.

In the following description and claims, the terms “computer program medium” and “computer readable medium” may be used to generally refer to media such as, but not limited to, removable storage drives, a hard disk installed in hard disk drive, and the like. These computer program products may provide software to a computer system. Embodiments of the invention may be directed to such computer program products.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory. A “computing platform” may comprise one or more processors.

A non-transitory computer readable medium includes, but is not limited to, a hard drive, compact disc, flash memory, volatile memory, random access memory, magnetic memory, optical memory, semiconductor based memory, phase change memory, optical memory, periodically refreshed memory, and the like; however, the non-transitory computer readable medium does not include a pure transitory signal per se.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied. Thus, the present invention is not limited to any particular tangible means of implementation.

The present invention will now be described in detail with reference to embodiments thereof as illustrated in the accompanying drawings.

There are various types of neurological monitoring methods and systems that may be provided by preferred embodiments of the present invention. In one embodiment of the present invention, the neurological monitoring method allows a user to monitor a neurological organ while recovering from an injury that is related to the neurological organ. In other embodiments, the neurological monitoring method may allow a user to recover from the injury, map the neurological organ, and study the neurological organ. The neurological organ may include, without limitation, the brain, the spine, and the nervous system. However, in other embodiments, any organ that produces a physical or electrophysiological signal may be monitored by the neurological monitoring method. The injury may include, without limitation, a mild traumatic brain injury, a concussion, a hemorrhage, and a headache. In some embodiments, the method may monitor a signal generated by the neurological organ. The signal may include, without limitation, electrophysiological signals, electrical pulses, voltage fluctuations from ionic current flows, and frequencies of Hertz per second.

In one embodiment of the present invention, a sensor portion may receive the signal. The sensor portion may join in proximity to an affected area to provide a more accurate reading of the injury. The sensor portion may include, without limitation, electrodes, EEG recording nets, and circuits. The sensor portion may be configured to transmit readings from the signal to a data storage portion. The data storage portion may be remotely located in relation to the sensor portion. The data storage portion may include, without limitation, a database, a processor, a network, and a cloud. In some embodiments, the data storage portion may be operable to transform the signal from one form to another.

In one embodiment of the present invention, the data storage portion may transmit the signal to a display portion. The display portion may include, without limitation, a mobile electronic device, a smart phone, a computer monitor, and a tablet. The display portion may display the signal that correlates to the neurological organ. The display portion may include a signal parameter that displays a high range and a low range acceptable for the signal of the neurological organ. In some embodiments, the display portion may alert the user if the signal reaches a predetermined level. In this manner, the user may be informed if the neurological organ is not functioning properly, is functioning properly, is healing, or requires additional attention. The display portion may further include the identification of the user. The display portion may further include a date and time stamp indicating the last period that the user was physically examined, or the last time the signal was monitored. A follower may also receive the signal from a different display portion. The follower may include, without limitation, a health care professional and a family member. In one embodiment, the follower may also be alerted if the signal reaches a predetermined level to know if the neurological organ is not functioning properly. In this manner, the follower may assist the user. The follower may monitor a plurality of users from the display portion. In yet another embodiment, the follower and the user may interchange messages pertinent to the neurological monitoring method through the respective display portions. In yet another embodiment, the follower may set a predetermined parameter for the signal. If the parameter is exceeded, the display portion may alert the follower to take corrective actions.

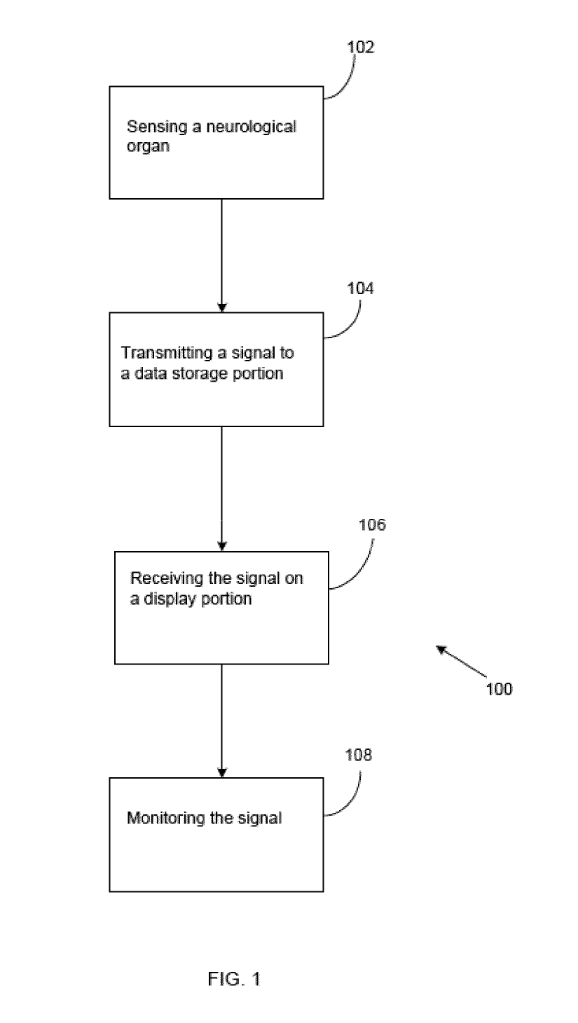

FIG. 1 illustrates a flowchart diagram of the steps for an exemplary neurological monitoring method 100, in accordance with an embodiment of the present invention. In the present embodiment, the neurological monitoring method may include a Step 102 of sensing the neurological organ with at least one sensor portion. Those skilled in the art, in light of the present teachings will recognize that the initial days and weeks of a recovery period for a concussion or mild traumatic brain injury require the avoidance of physical activity, cognitive activity, and neuro stimulation which increase neuro-electrophysiological activity. The increased activities may increase blood flow in a concussed brain causing swelling and intracranial pressure. Furthermore, for a period of minutes to days to weeks after a concussion, the brain may be especially vulnerable to changes in intracranial pressure, blood flow, and anoxia. Therefore, monitoring the neurological organ in the early stages may be vital to a proper recovery from injury.

In one embodiment of the present invention, the at least one sensor portion may join in proximity to an affected area to provide a more accurate reading of the injury. The sensor portion may include, without limitation, electrodes, EEG recording nets, and circuits. In one embodiment, sensing the neurological organ may include, without limitation, recording electrical activity along the scalp, with electrodes positioned on the scalp. Various sensing methods may be utilized including, without limitation, electroencephalography (EEG), electromyography (EMG), electrooculography (EOG), functional magnetic resonance imaging (fMRI), positron emission tomography, magnetoencephalography, nuclear magnetic resonance spectroscopy, electrocorticography, and single-photon emission computed tomography. The sensor portion may be configured to transmit readings from the signal to the data storage portion.

In one embodiment of the present invention, the neurological monitoring method allows a user to monitor a neurological organ while recovering from an injury that is related to the neurological organ. In other embodiments, the neurological monitoring method may allow a user to recover from the injury, map the neurological organ, and study the neurological organ. The neurological organ may include, without limitation, the brain, the spine, and the nervous system. However, in other embodiments, any organ that produces a physical or electrophysiological signal may be monitored by the neurological monitoring method. The injury may include, without limitation, a mild traumatic brain injury, a concussion, a hemorrhage, and a headache. In some embodiments, the method may monitor a signal generated by the neurological organ. The signal may include, without limitation, electrophysiological signals, electrical pulses, voltage fluctuations from ionic current flows, and frequencies of Hertz per second.

In one embodiment of the present invention, Step 104 may include transmitting the signal to a data storage portion. Suitable conventional communication protocols used by the system may include, without limitation IEEE 802.15 (Bluetooth), or proprietary 802.15.x protocols such as 802.15.4 (Zigbee) or other such proprietary protocols such as 2.4 GHz ISM band used by ANT by Dynastream Innovations Inc. The system may also use, without limitation, protocols such as WiFi (802.11) or conventional cellular technologies such as TCPIP or UDP. The sensor portion may be configured to transmit readings from the signal to a data storage portion. The data storage portion may be remotely located in relation to the sensor portion. The data storage portion may include, without limitation, a database, a processor, a network, and a cloud. In some embodiments, the data storage portion may be operable to transform the signal from one form to another. In one embodiment of the present invention, Step 106 may include receiving the signal on a display portion. The display portion may include, without limitation, a mobile electronic device, a smart phone, a personal computer, and a tablet. The display portion may display the signal that correlates to the neurological organ. The display portion may include a signal parameter that displays a high range and a low range acceptable for the signal of the neurological organ. In some embodiments, the display portion may alert the user if the signal reaches a predetermined level. In this manner, the user may be informed if the neurological organ is not functioning properly, is functioning properly, is healing, or requires additional attention. The display portion may further include the identification of the user. The display portion may further include a date and time stamp indicating the last period that the user was physically examined, or the last time the signal was monitored. A follower may also receive the signal from a different display portion. The follower may include, without limitation, a health care professional and a family member.

In one embodiment of the present invention, Step 108 may include monitoring the resultant signal from the neurological organ. The display portion may allow the user and the follower to simultaneously monitor the status of the neurological organ. Those skilled in the art will recognize that the display portion may be configured to display only pertinent information regarding the signal and the damaged neurological organ. In some embodiments, the follower may receive and view the signal from a different display portion simultaneously with the user. The follower may include, without limitation, a health care professional and a family member. In one embodiment, the follower may also be alerted if the signal reaches a predetermined level to know if the neurological organ is not functioning properly. In this manner, the follower may assist the user. The follower may monitor a plurality of users from the display portion. In yet another embodiment, the follower and the user may interchange messages pertinent to the neurological monitoring method through the respective display portions. In yet another embodiment, the follower may set a predetermined parameter for the signal. If the parameter is exceeded, the display portion may alert the follower to take corrective actions.

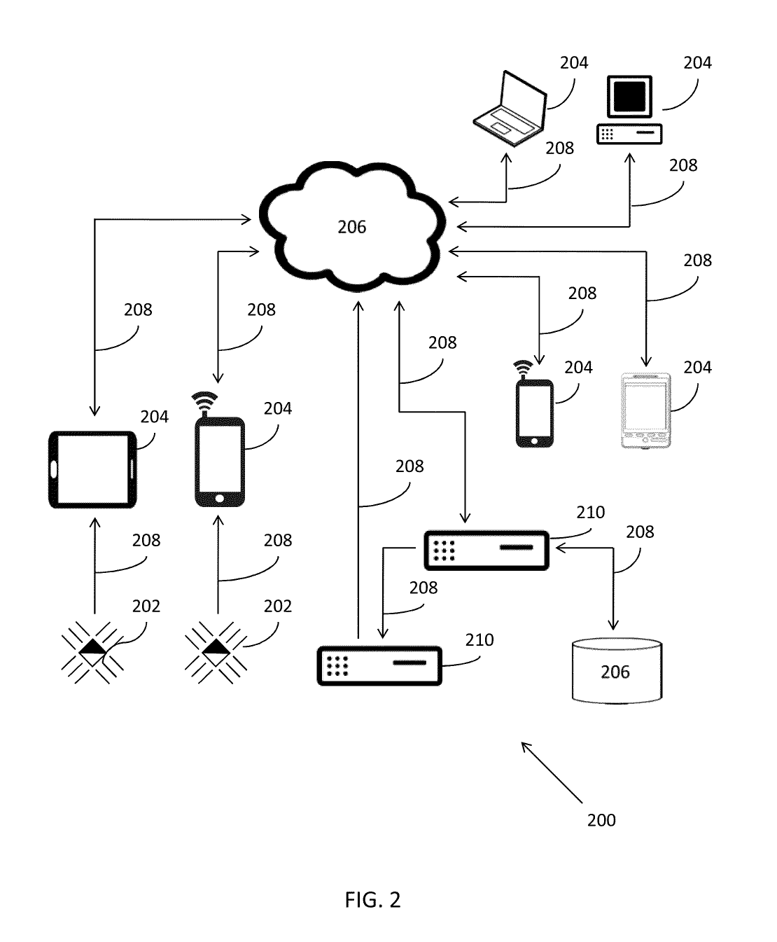

FIG. 2 illustrates a block diagram for an exemplary neurological monitoring system 200, in accordance with an embodiment of the present invention. In the present embodiment, the neurological monitoring system may include numerous systems and components working in conjunction to provide a system for monitoring the neurological organ. The sensor portion 202 may join in proximity to the neurological organ to sense for electrical signals 208 that the neurological organ emits. Those skilled in the art, in light of the present teachings will recognize that voltage fluctuations resulting from ionic current flows within the neurons of the brain emit to form signals. The signal may include, without limitation, electrophysiological signals, electrical pulses, voltage fluctuations from ionic current flows, and frequencies of Hertz per second. A display portion 204 may provide a visual confirmation and analysis of the signal. The sensor portion, which may be configured to include the display portion, may be configured to transmit readings from the signal to a data storage portion 206. The data storage portion may be remotely located in relation to the sensor portion. The data storage portion may include, without limitation, a database, a processor, a computing device such as a personal computer, smart phone and a tablet, a network, and a cloud. In some embodiments, the data storage portion may be operable to transform the signal from one form to another.

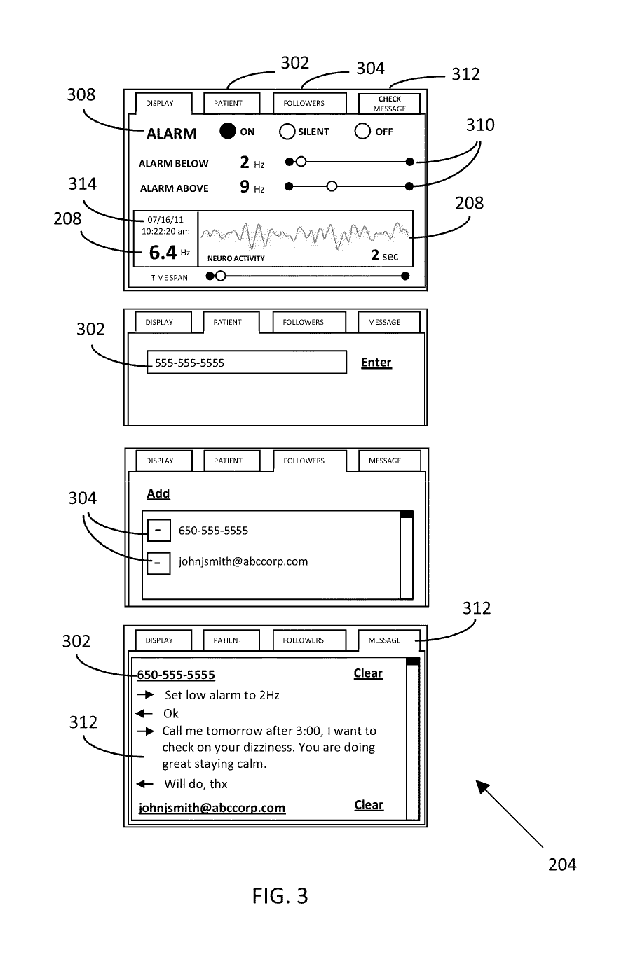

FIG. 3 illustrates a top view of an exemplary display portion for a user, in accordance with an embodiment of the present invention. In the present embodiment, the user may monitor the signals emanating from the neurological organ in real time on the display portion. The display portion may include, without limitation, a mobile electronic device, a smart phone, a personal computer, and a tablet. The display portion may display the signal that correlates to the neurological organ. The display portion may include a signal parameter 310 that displays a high range and a low range acceptable for the signal of the neurological organ. In some embodiments, the display portion may include an alert portion 308 that alerts a user if the signal reaches a predetermined level 310. In this manner, the user may be informed if the neurological organ is not functioning properly, is functioning properly, is healing, or requires additional attention. The display portion may further include the identification of a user 302. The display portion may further include a date and time portion 314 indicating the last period that the user was physically examined, or the last time the signal was monitored. The display portion may further include a message portion 312 so that a user and a follower 304 may communicate with each other.

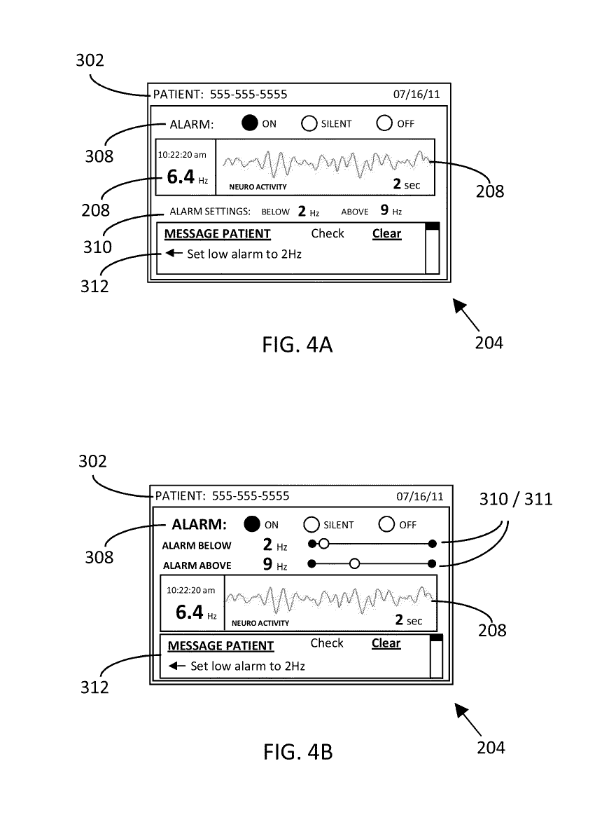

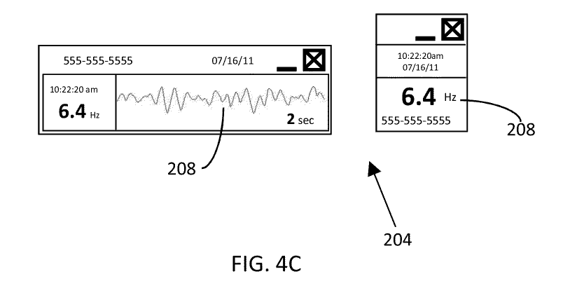

FIGS. 4A, 4B, and 4C illustrate a top view of an exemplary display portion for a follower, wherein FIG. 4A illustrates an exemplary alert portion set for the follower and the user, FIG. 4B illustrates an exemplary alert portion set by the follower to track the user, and FIG. 4C illustrates an exemplary desktop display portion used by the follower, in accordance with an embodiment of the present invention. In the present embodiment, the follower may receive the signal from a different display portion simultaneously with the user. In this manner, the follower may better monitor the neurological organ's status. The follower may set the alert portion to alert if the parameters are exceeded 310. The follower may further set the alert portion so that only the follower is aware of an irregularity 311. In this manner, the user may not be stressed. In one embodiment, the follower may also be alerted if the signal reaches a predetermined level to know if the neurological organ is not functioning properly. In this manner, the follower may assist the user. The follower may monitor a plurality of users from the display portion. In yet another embodiment, the follower and the user may interchange messages pertinent to the neurological monitoring method through the respective display portions 312. In yet another embodiment, the follower may set a predetermined parameter for the signal 310. If the parameter is exceeded, the display portion may alert the follower to take corrective actions.

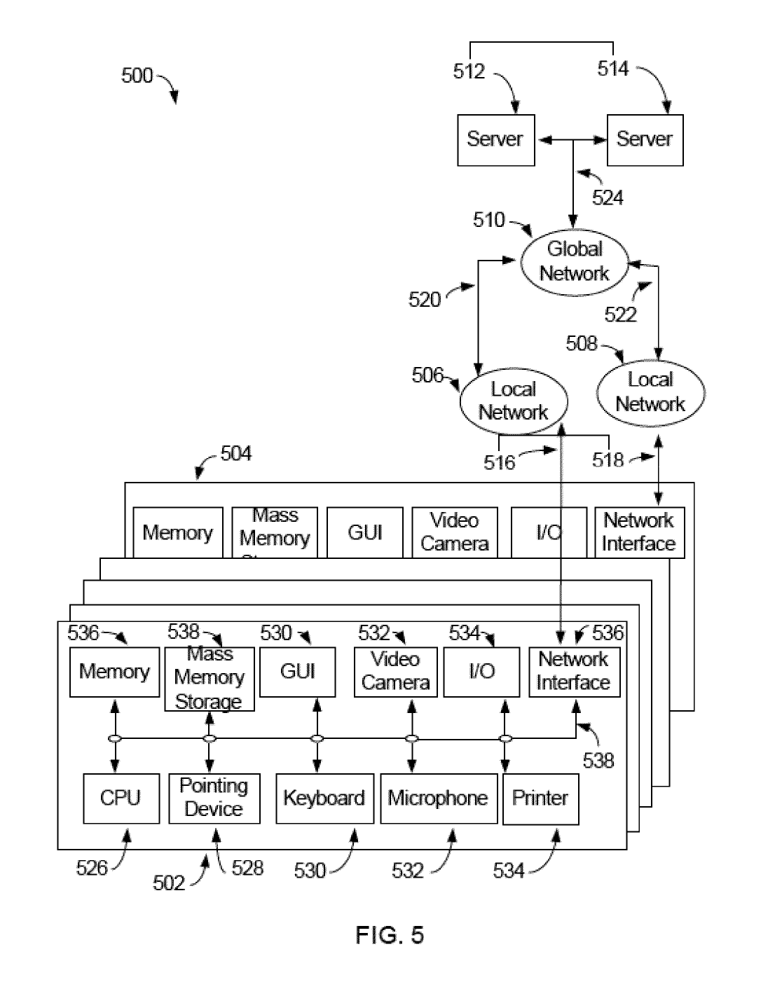

FIG. 5 illustrates a typical computer system that, when appropriately configured or designed, can serve as a computer system in which the invention may be embodied, in accordance with an embodiment of the present invention. In the present embodiment, the computer system 500 may include any number of processors 502 (also referred to as central processing units, or CPUs) that are coupled to storage devices including primary storage 506 (typically a random access memory, or RAM), primary storage 504 (typically a read only memory, or ROM). CPU 502 may be of various types including microcontrollers (e.g., with embedded RAM/ROM) and microprocessors such as programmable devices (e.g., RISC or SISC based, or CPLDs and FPGAs) and unprogrammable devices such as gate array ASICs or general purpose microprocessors. As is well known in the art, primary storage 504 acts to transfer data and instructions uni-directionally to the CPU and primary storage 506 is used typically to transfer data and instructions in a bi-directional manner. Both of these primary storage devices may include any suitable computer-readable media such as those described above. A mass storage device 508 may also be coupled bi-directionally to CPU 502 and provides additional data storage capacity and may include any of the computer-readable media described above. Mass storage device 508 may be used to store programs, data and the like and is typically a secondary storage medium such as a hard disk. It will be appreciated that the information retained within the mass storage device 508, may, in appropriate cases, be incorporated in standard fashion as part of primary storage 506 as virtual memory. A specific mass storage device such as a CD-ROM 514 may also pass data uni-directionally to the CPU.

CPU 502 may also be coupled to an interface 538 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 502 optionally may be coupled to an external device such as a database or a computer or telecommunications or internet network using an external connection as shown generally at 536, which may be implemented as a hardwired or wireless communications link using suitable conventional technologies. With such a connection, it is contemplated that the CPU might receive information from the network, or might output information to the network in the course of performing the method steps described in the teachings of the present invention.

All the features or embodiment components disclosed in this specification, including any accompanying abstract and drawings, unless expressly stated otherwise, may be replaced by alternative features or components serving the same, equivalent or similar purpose as known by those skilled in the art to achieve the same, equivalent, suitable, or similar results by such alternative feature(s) or component(s) providing a similar function by virtue of their having known suitable properties for the intended purpose. Thus, unless expressly stated otherwise, each feature disclosed is one example only of a generic series of equivalent, or suitable, or similar features known or knowable to those skilled in the art without requiring undue experimentation.

Having fully described at least one embodiment of the present invention, other equivalent or alternative methods of implementing a method for monitoring neurological activities according to the present invention will be apparent to those skilled in the art. Various aspects of the invention have been described above by way of illustration, and the specific embodiments disclosed are not intended to limit the invention to the particular forms disclosed. The particular implementation of the method for monitoring neurological activities may vary depending upon the particular context or application. By way of example, and not limitation, the method for monitoring neurological activities described in the foregoing were principally directed to monitoring brain activity; however, similar techniques may instead be applied to other organs of the body, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

What is claimed is:

1. One or more computer storage media storing computer-usable instructions, that when used by one or more computing devices, cause the one or more computing devices to perform a method comprising the steps of:
(a) sensing a neurological organ;
(b) transmitting a signal to a data storage portion;
(c) receiving said signal on a display portion; and
(d) monitoring said signal.

2. The method of claim 1, in which step (a) further comprises utilizing at least one sensor to record activity of said neurological organ.

3. The method of claim 2, in which said neurological organ comprises a brain.

4. The method of claim 3, in which said at least one sensor comprises at least one electroencephalography electrode.

5. The method of claim 4, in which said electroencephalography electrode is operable to generate a brainwave frequency, said brainwave frequency comprising Hertz.

6. The method of claim 5, in which said brainwave frequency comprises a graphical wave form display.

7. The method of claim 1, in which step (b) further comprises transferring said signal with a conventional communication protocol, said conventional communication protocol comprising IEEE 802.15 Bluetooth.

8. The method of claim 7, in which step (c) further comprises displaying said signal on a display portion.

9. The method of claim 8, in which step (c) further comprises displaying said signal that correlates to said neurological organ.

10. The method of claim 9, in which said signal comprises a signal parameter, said signal parameter comprising a high range and a low range.

11. The method of claim 10, in which said signal parameter comprises an alert portion, said alert portion being operable to monitor concussion recovery, said alert portion comprising audible, visible and/or physical indicators.

12. The method of claim 11, in which said display portion comprises a portable electronic device.

13. The method of claim 1, in which said display portion comprises displaying via a non-cloud based application.

14. The method of claim 1, in which said monitoring occurs via a portable electronic device having wireless connectivity.

15. The method of claim 14, in which step (d) further comprises a cloud based application, said cloud based application being operable to support at least one invited follower to view or interact with said display portion of said user.

16. The method of claim 15, wherein said follower manipulates said signal parameter.

17. The method of claim 16, in which step (d) further comprises monitoring a real time frequency, said real time frequency comprising about 0.01 to 10 seconds, said real time frequency further comprising a periodic average, said periodic average comprising about 0.001 to 300 seconds.

18. The method of claim 17, in which said user and said follower monitor the display portion simultaneously.

19. A system for monitoring comprising:
means for sensing a neurological organ;
means for transmitting a signal to a data storage portion;
means for receiving said signal on a display portion; and
means for monitoring said signal.

20. A non-transitory program storage device readable by a machine tangibly embodying a program of instructions executable by the machine to perform a method for monitoring, the storage device comprising:
(a) computer code for sensing a neurological organ, said neurological organ comprising a brain;
(b) computer code for transmitting a signal to a data storage portion, said signal being configured to correlate to said neurological organ, said signal comprising a signal parameter, said signal parameter comprising an alert portion;
(c) computer code for receiving said signal on a display portion, said display portion comprising a portable electronic device; and
(d) computer code for monitoring said signal.
